(19) United States
(12) Patent Application Publication
(10) Pub. No.: US 2017/0030930 A1
(43) Pub. Date: Feb. 2, 2017

(54) DIAGNOSIS AND TREATMENT OF TAUOPATHY AND CHRONIC TRAUMATIC ENCEPHALOPATHY

(71) Applicant: Government of the United States, as represented by the Secretary of the Army, Silver Spring, MD (US)

(72) Inventors: Joseph B. Long, Clarksville, MD (US); Peethambaran Arun, Clarksburg, MD (US)

(73) Assignee: The Government of the United States, as represented by the Secretary of the Army, Silver Spring, MD (US)

(21) Appl. No.: 15/292,936

(22) Filed: Oct. 13, 2016

Related U.S. Application Data
(63) Continuation of application No. PCT/US2015/000045, filed on Apr. 14, 2015.
(60) Provisional application No. 61/997,050, filed on Apr. 15, 2014.

(51) Int. Cl. G01N 33/68; G01N 33/573; C12N 9/16; A61K 38/46; A61K 9/00

(52) U.S. Cl. CPC G01N 33/6896; C12Y 301/03001; A61K 38/465; A61K 9/0043; C12N 9/16; G01N 33/573; G01N 2333/916; G01N 2800/28

(57) ABSTRACT

A method of diagnosing TBI-induced tauopathy/chronic traumatic encephalopathy (CTE) by obtaining control samples from a control patients that have not been exposed to TBI and recording a normal range of tissue non-specific alkaline phosphatase (TNAP) or total alkaline phosphatase (AP) activity. Then obtaining samples from object patients that have been exposed to TBI. Comparing the biomarker, TNAP/AP, levels of said object patients to the controls. Then determining if the object patient has been exposed to TBI if the TNAP/AP levels are decreased below the normal range. Treating the patient by increasing the level of TNAP enzyme in the brain to within a normal range or modifying the TNAP enzyme activity so that it regains normal activity.

This application claims priority from U.S. Provisional Application Ser. No. 61/997,050 filed on Apr. 15, 2014.

### GOVERNMENT INTEREST

The invention described herein may be manufactured, used and licensed by or for the U.S. Government.

### BACKGROUND OF THE INVENTION

1. Field of the Invention

The present invention relates to the field of diagnosis and treatment of traumatic brain injury.

2. Brief Description of Related Art

The incidence of traumatic brain injury (TBI) on the battlefield has increased tremendously during recent conflicts due to the widespread use of improvised explosive devices and other modern explosive weaponries. Exposure to blast has been described as the major cause of TBI and associated disabilities in the recent wars in Iraq and Afghanistan (Magnuson et al., 2012). Although several biochemical and histopathological changes have been documented in the central nervous system after blast exposure (Kocsis & Tessler, 2009; Saljo et al, 2002; Cernak et al, 2001b; Cernak et al, 2001a; Svetlov et al, 2010; Long et al, 2009; Cernak et al, 2011; Wang et al, 2011), the potentially complex pathophysiological mechanisms triggering long-term neurobehavioral abnormalities are still not well understood, which has hampered the development of effective countermeasures and diagnostic approaches. Recent studies indicate that chronic traumatic encephalopathy (CTE), a tau protein-linked neurodegenerative disorder observed in athletes with multiple concussions, shares clinical symptoms and neuropathological characteristics with victims of blast exposure (Goldstein et al, 2012). In particular, phosphorylated Tau protein (pTau) neuropathology with perivascular neurofibrillary degeneration, a distinct feature of CTE was observed in the postmortem brain of blast exposed victims, amateur athletes, and in the brains of mice exposed to blast overpressure using shock tube and suggested that hyperacceleration of head plays an important role in the development of CTE (Goldstein et al, 2012). Phosphorylation of Tau protein disrupts microtubule assembly in neurons which can result in tauopathy and the formation of neurofibrillary tangles seen in neurodegenerative disorders such as Alzheimer's disease (AD) (Hanger et al, 1991; Iqbal et al, 1994; Wang et al, 1996). Dephosphorylation of pTau is critical to prevent tauopathy and to restore microtubule assembly for neuroregeneration.

### SUMMARY OF THE INVENTION

Phosphorylation of Tau inhibits microtubule assembly in the neurons leading to neurofibrillary tangle formation, neurodegeneration, tauopathy and CTE.

Tissue non-specific alkaline phosphatase (TNAP) is a critical enzyme involved in the dephosphorylation of pTau and decrease in its activity can lead to accumulation of pTau, tauopathy and CTE.

Blast exposure as well as head impact acceleration in rats leads to decreased expression and activity of TNAP in different regions of the brain. The decrease in TNAP activity was associated with accumulation of pTau in different regions of the brain.

The decreased activity of TNAP in the brain after blast exposure as well as after head impact in rats is associated with a decreased activity of alkaline phosphatase (AP) in the plasma which can potentially be used as a biomarker of tauopathy/CTE.

### BRIEF DESCRIPTION OF THE DRAWINGS

FIG. 1A is a digital photograph of a Western blotting showing the expression of pTau in different brain regions at 6 hr and 24 hr after blast or weight drop with representative figures from two rats out of four in each group are shown;

FIG. 1B is a graph showing densitometry analysis of the ratio of band intensities of pTau and β-actin at 6 hours. Values are expressed as mean±SD. *p<0.05;

FIG. 1C is a graph showing densitometry analysis of the ratio of band intensities of pTau and β-actin at 24 hours. Values are expressed as mean±SD. *p<0.05;

FIG. 2A is a graph showing activity of TNAP in different brain regions at 6 hr after blast or weight drop. Values are expressed as mean±SD. n=4, *p<0.05, **p<0.01;

FIG. 2B is a graph showing activity of TNAP in different brain regions at 24 hr after blast or weight drop. Values are expressed as mean±SD. n=4, *p<0.05, **p<0.01;

FIG. 3. is a graph showing activity of alkaline phosphatase in the plasma at different intervals after blast or weight drop. Values are expressed as mean±SD. n=4, * p<0.05, **p<0.10;

FIG. 4A is a digital photo graph of a Western blotting showing the expression of TNAP in different brain regions at 6 hr and 24 hr after blast or weight drop with representative figures from two rats out of four in each group are shown;

FIG. 4B is a graph showing densitometry analysis showing the ratio of band intensities of TNAP and β-actin at 6 hours with values are expressed as mean±SD*p<0.05;

FIG. 4C is a graph showing densitometry analysis showing the ratio of band intensities of TNAP and β-actin at 24 hours with values are expressed as mean±SD*p<0.05;

FIG. 5A is a schematic representation of the shock tube used to expose rats to blast overpressure waves; and

FIG. 5B is a graph showing the pressure profile generated inside the shock tube of FIG. 5A where the animals were kept.

### DETAILED DESCRIPTION

Recent studies indicate that chronic traumatic encephalopathy (CTE), a tau protein-linked neurodegenerative disorder observed in athletes with multiple concussions, shares clinical symptoms and neuropathological characteristics with victims of blast exposure (Goldstein et al, 2012). In particular, phosphorylated Tau protein (pTau) neuropathology with perivascular neurofibrillary degeneration, a distinct feature of CTE was observed in the postmortem brain of blast exposed victims, amateur athletes, and in the brains of mice exposed to blast overpressure using shock tube and suggested that hyperacceleration of head plays an important role in the development of CTE (Goldstein et al, 2012). Phosphorylation of Tau protein disrupts microtubule assembly in neurons which can result in tauopathy and the formation of neurofibrillary tangles seen in neurodegenerative disorders such as Alzheimer's disease (AD) (Hanger et al, 1991; Iqbal et al, 1994; Wang et al, 1996). Dephosphorylation of pTau is critical to prevent tauopathy and to restore microtubule assembly for neuroregeneration.

Tissue non-specific alkaline phosphatase (TNAP) plays a major role in the brain by dephosphorylating pTau in neurons (Hanger et al, 1991; Iqbal et al, 1994; Wang et al, 1996). Paired helical filaments and Tau protein isolated from AD patients' brains formed a microtubule assembly with tubulin in vitro only after treatment with alkaline phosphatase or protein phosphatase-2A, 2B and -1, suggesting that Tau protein in the paired helical filaments of neurons in AD brain is hyperphosphorylated which prevents microtubule assembly (Hanger et al, 1991; Iqbal et al, 1994; Wang et al, 1996). Alkaline phosphatase showed significantly higher activity in dephosphorylating pTau compared to other protein phosphatases studied (Wang et al, 1996).

A number of studies indicate that accumulation of amyloid precursor protein (APP) and 3-amyloid peptides induces the phosphorylation of Tau and leads to microtubule disassembly, an accepted neuropathological mechanism of AD (Greenberg et al, 1994; Le et al, 1997; Busciglio et al, 1995). Activation of mitogen-activated protein kinase by accumulated APP has been described as a mechanism of phosphorylation of Tau protein (Greenberg et al, 1994). In a hybrid septal cell line, treatment with aggregated 3-amyloid peptide resulted in accumulation of pTau and paired helical filaments and alkaline phosphatase treatment abolished the effect (Le et al, 1997) emphasizing the role of TNAP in preventing Tau phosphorylation.

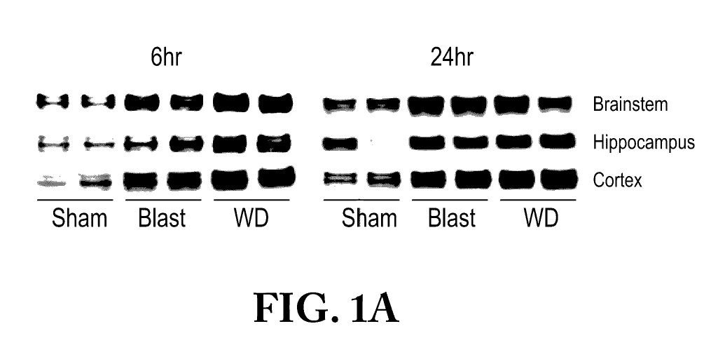

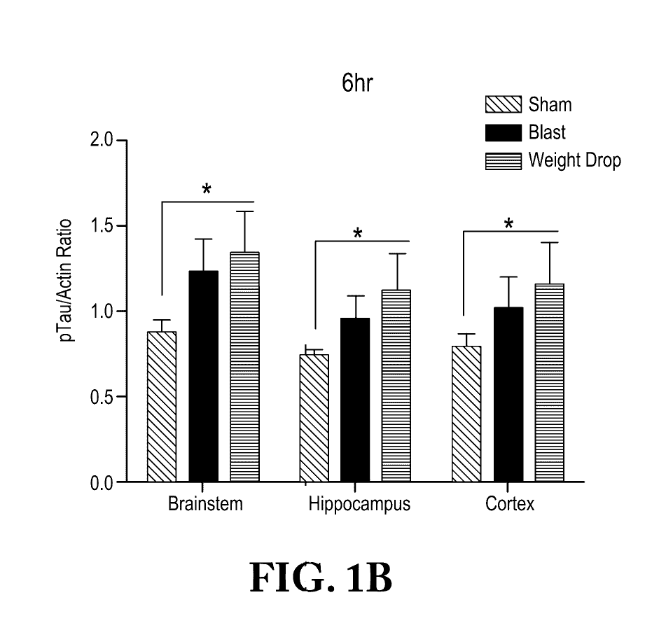

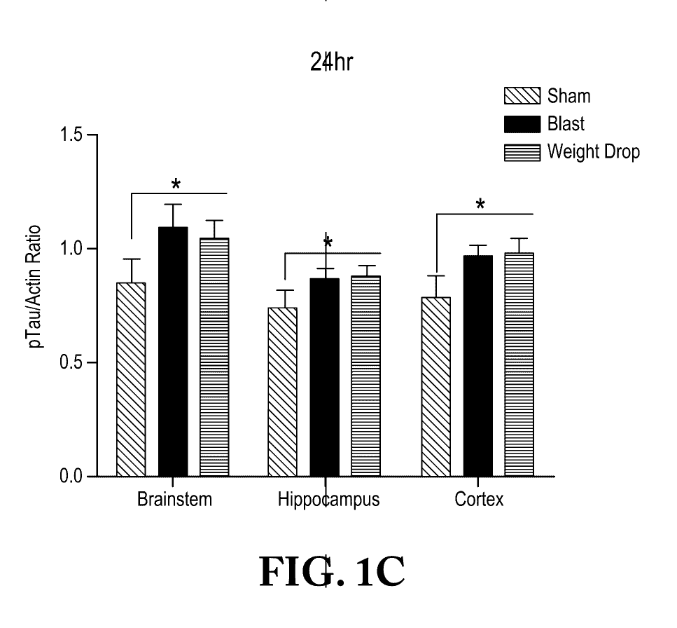

Our studies in the rat using a shock tube model of blast-induced TBI and diffuse brain injury induced by head impact with a slightly modified Marmarou weight drop model (Marmarou et al, 1994) revealed pTau accumulation in different regions of the brain as early as 6 h post-injury and further increases by 24 h (FIGS. 1 A, 1B and 1C).

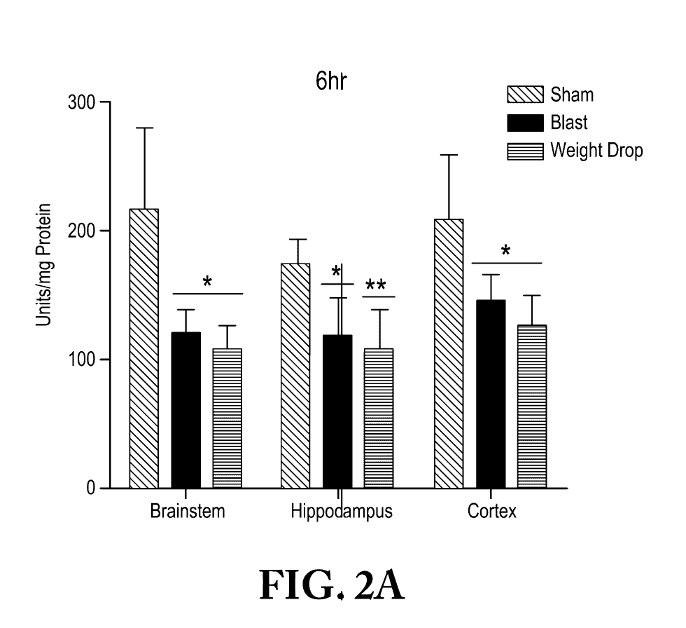

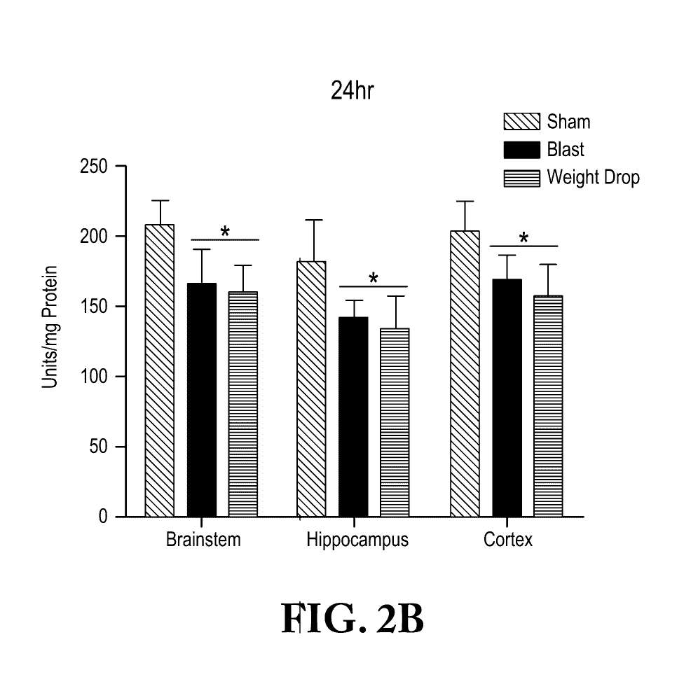

The extent of phosphorylation of Tau varies in different regions of the brain after the insults. Measurement of TNAP activity showed a significant decrease in different brain regions at 6 and 24 hr after either blast exposure or weight drop. The results obtained indicate that brain injury after blast or weight drop causes significant decrease in the activity of TNAP at both 6 and 24 hr post-injury. (FIGS. 2A and 2B) At 6 hr, blast exposure resulted in 44.8%, 32.5% and 31.4% decrease in TNAP activity in brainstem, hippocampus and cortex respectively where as in the case of weight drop, the decreases were 50.6%, 38.9% and 40.4% respectively. At 24 hr, blast exposure caused 20.2%, 22.9% and 17.7% decrease in TNAP activity in brainstem, hippocampus and cortex respectively where as in the case of weight drop, the decreases were 23.3%, 26.8% and 22.6% respectively. Weight drop resulted in more decrease in TNAP activity compared to blast exposure in different brain regions at 6 and 24 hr, despite any statistical significance. Additionally, the decrease in TNAP activity was maximum at 6 hr compared to 24 hr post-injury. (FIGS. 2A and 2B).

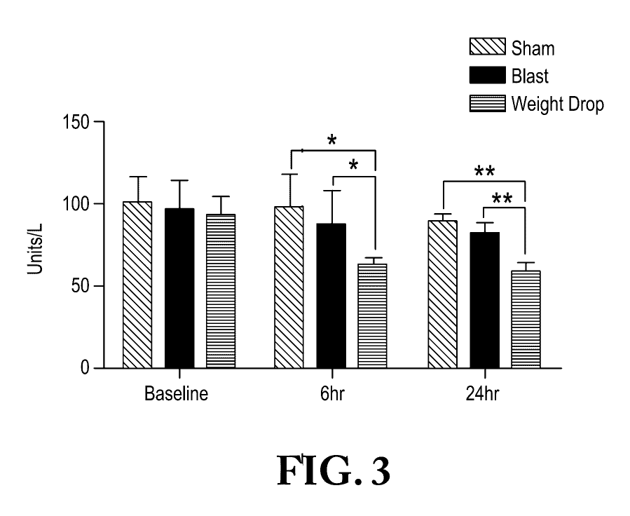

Total alkaline phosphatase (AP) activity in the plasma showed a significant decrease after weight drop (FIG. 3). Blast exposure also resulted in a decrease in TNAP activity compared to sham control, despite any statistical significance. Alkaline phosphatase activity in the plasma at different intervals after blast exposure or weight drop was significantly decreased at 6 and 24 hr. Plasma alkaline phosphatase activity was significantly less in the animals subjected to weight drop compared blast exposed animals. Weight drop caused 32.3% and 36.7% decrease in TNAP activity in the plasma at 6 and 24 hr respectively. (FIG. 3).

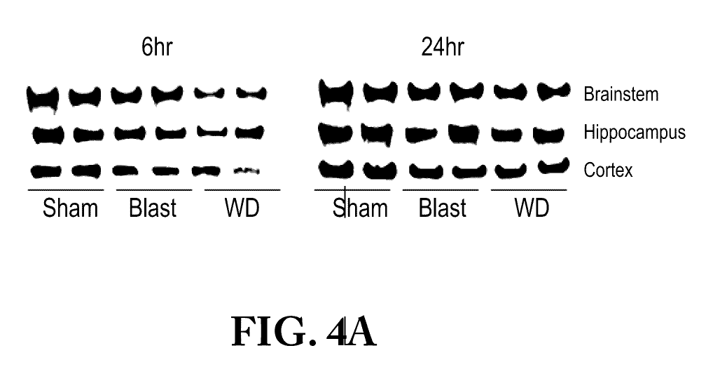

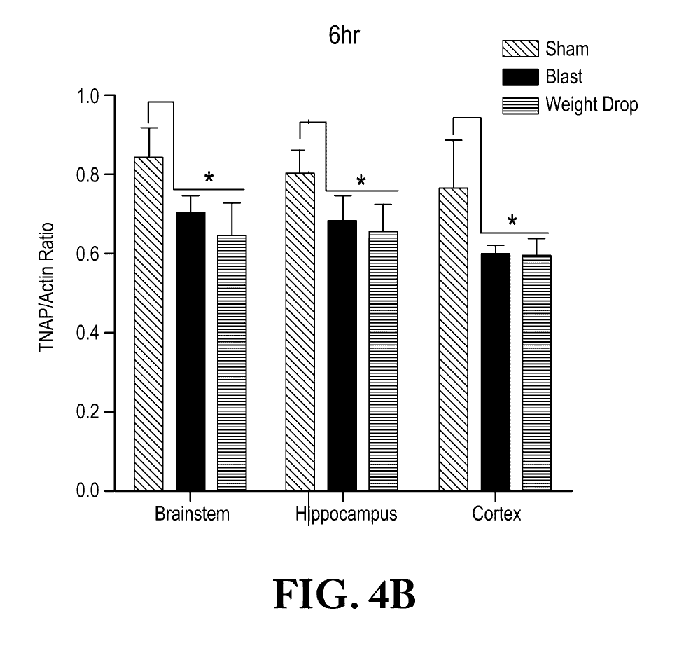

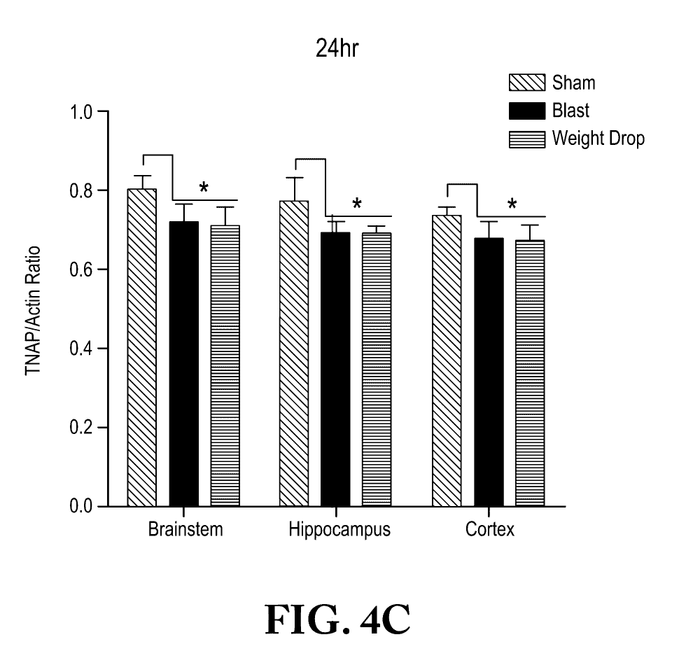

Western blot analysis using monoclonal antibodies against TNAP showed decreased levels of TNAP expression in different regions of the brain at 6 and 24 hr after blast exposure or weight drop with the maximum decrease after weight drop (FIGS. 4A, 4B and 4C). The decrease in the expression of TNAP in different brain regions was comparable after blast exposure and weight drop. The decrease in the expression of TNAP was more at 6 hr compared 24 hr even though the differences were statistically not significant.

The decrease in alkaline phosphatase enzyme activity in the plasma was significantly more after weight drop compared to blast exposure at early time points and a correspondingly higher accumulation of pTau in brain regions was observed in the weight drop model, suggesting the potential use of TNAP or AP as a marker for the diagnosis and prognosis of blast-induced tauopathy/CTE. The results also suggest that a significant amount of the alkaline phosphatase in the blood originates in the brain since the weight drop model has injury focused only to the head/brain. These observations suggests that the decreased levels/activity of TNAP in the brain immediately after blast exposure might be responsible for the accumulation of pTau after blast exposure as well as weight drop which can lead to chronic neurodegeneration, tauopathy and CTE.

### Methods Used

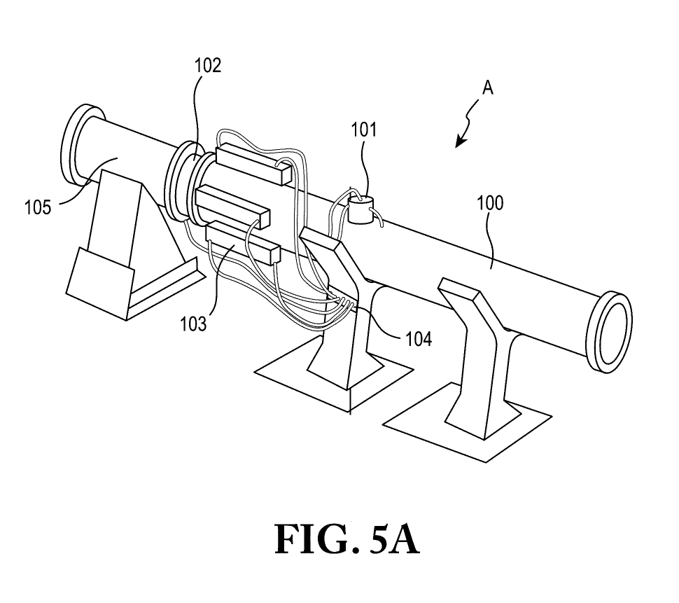

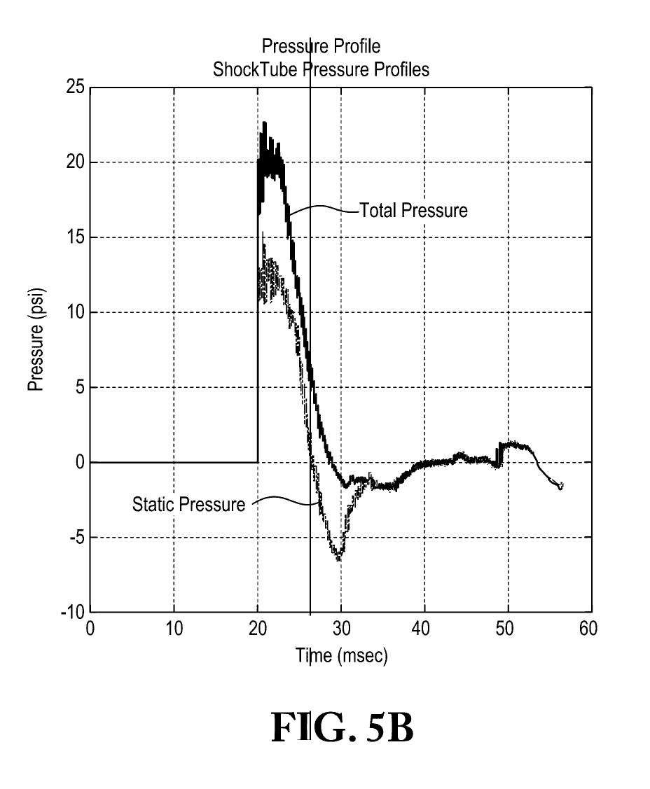

Blast TBI model: Male Sprague Dawley rats (300-350 g body weight, Charles River Laboratories) were anesthetized with isofluorane and placed in a transverse prone position 2.5 ft inside of a 15 ft long compressed air-driven shock tube (FIG. 5A) described earlier (Long et al, 2009). The tube A consists of an expansion chamber 100, a hydraulic control 101, hydraulic control manifold 104, hydraulic arm 103, compression chamber 105 and a Mylar diaphragm placement 102.

The animals were exposed to a single blast overpressure of 19 psi (133 kPa). At 6 hours and 24 hours after blast exposure, the animals were euthanized and collected brain and blood plasma. The brains were dissected into cortex, brainstem and hippocampus. The brain regions and plasma were stored at −80° C. until analyses.

The pressure profile generated inside the shock tube where the rats were positioned is shown in FIG. 5B.

Head impact/acceleration model using weight drop: As originally described by Marmarou et al. (Marmarou et al, 1994), the injury device consisted of a 2.5 m long Plexiglas tube with a 19 mm inner diameter clamped to a ringstand. The heads of the isoflurane-anesthetized rats were covered with a helmet made of Mylar sheet to prevent any skull fracture during weight drop. The rats were positioned in a prone position on a 12×12×43 cm foam bed (Type E manufactured by Foam to Size, Inc., Ashland, Va.) of known spring constant which is contained without compression within a Plexiglas frame. After securing the rat to the foam bed, the tube was positioned directly over the rat's head and the cap was adjusted so that the striking plate was horizontal and parallel to the impacting face of the falling weight. Brain injury was produced by dropping brass weight (500 g) from a predetermined height (150 cm). Rebound impact by the weight was prevented by sliding the foam bed and rat away from the tube immediately after impact/acceleration.
Measurement of TNAP activity in the brain: Activity of TNAP in different regions of the brain was carried out using alkaline phosphatase assay kits from Randox Laboratories (Kearneysville, W.V.) according to manufacturer's instructions. Briefly, 20% brain homogenates was made in T-Per tissue protein extraction buffer (Pierce Chemicals Co, Rockford, Ill.) containing protease inhibitors using a Sonifier. After centrifugation at 13000 g for 5 min, the supernatants were collected. For activity assay, 5 μl each of the above supernatants was added into the wells of a 96 well assay plate followed by addition of 200 μl of the assay mixture. The optical density at 405 nm was measured immediately and every 1 min for 5 min. The increase in optical density per minute was used for calculating the enzyme activity. Activity of TNAP was expressed in terms of total protein which was measured using Bio-Rad DC protein assay kit (BIO-RAD, Hercules, Calif.) according to manufacturer's instructions.
Measurement of total alkaline phosphatase activity in the plasma: Activity of total alkaline phosphatase in the plasma was determined using alkaline phosphatase assay kit from Randox Laboratories (Kearneysville, W.V.) according to manufacturer's instructions. Briefly, 5 μl each of plasma was added into the wells of a 96 well assay plate followed by addition of 200 μl of the assay mixture. The optical density at 405 nm was measured immediately and at 1 min intervals for 5 min. The increase in optical density per minute was used for calculating the activity. The enzyme activity was expressed in terms of volume of plasma.
Western blot analysis: The differential expression of TNAP and pTau in different regions of the brain at various intervals after brain injury was determined by Western blotting using monoclonal antibodies specific to TNAP and pTau. The extent of down-regulation or up-regulation of the proteins after the injuries was quantitated by densitometry using AlphaView v.1.3.0 software (Protein Simple, Santa Clara, Calif.).
Statistical analysis: Statistical analysis was carried out by analysis of variance (ANOVA) using GraphPad Prism (Version 5) software. Values were expressed as mean±standard deviation (SD). A p value less than 0.05 was considered significant.
Treatment for tauopathy/CTE:

Since it has been determined that the levels of pTau are elevated in the brain post injury and TNAP levels are decreased post injury, after diagnosis of injury, treatment should be administered. Treatment is in the form of increasing levels or activity of TNAP enzyme to the normal range. This can be accomplished by giving TNAP enzyme to a patient via nose to brain delivery using a nasal spray. Another way to increase the activity of the TNAP enzyme in the brain of a patient who has been injured is by intranasal administration of activators of the enzyme so that it will become enzymatically more active. We tested the intranasal administration f the enzyme and initial observations show that the enzyme reached the brain in the active form.

### Discussion:

In the present study, we have shown for the first time that the protein level and activity of TNAP in the brain decreases significantly after blast exposure or head impact acceleration and was associated with a significant increase in the phosphorylation of Tau protein. The decrease in TNAP activity in the brain after weight drop was more compared to blast exposure with a concomitant increase in the level of pTau in the brain after the weight drop induced injury suggesting that the deceased TNAP activity may be playing a role in the accumulation of pTau after the brain insults. The decrease in the activity of an enzyme could be due to the decreased level of the protein or due to an inhibition of the enzyme activity. Western bot analyses of the brain regions indicate that the TNAP protein level decreased after the brain insults. The decreased protein level of TNAP in the brain regions could be due to decreased synthesis or increased degradation of TNAP after the injury and further studies using messenger RNA levels are warranted to delineate the precise mechanism.

Despite any statistical significance, the level of pTau in the brain regions at 6 hr after the blast exposure was less compared to weight drop, whereas the levels were comparable at 24 hr.

The decrease in the activity of TNAP in the brain after weight drop was associated with a significant decrease in the activity of total alkaline phosphatase in the plasma. Compared to sham control, the animals exposed to blast also showed a decrease in the activity of alkaline phosphatase in the plasma despite any statistical significance. The alkaline phosphatase activity in the plasma of animals exposed to weight drop was significantly less compared to blast exposed animals suggesting that a significant amount of the alkaline phosphatase activity in the blood originates in the brain since the weight drop model has injury focused only to the head/brain.

### Conclusion

Brain injury after blast as well as head impact acceleration results in a significant decrease in the expression and activity of TNAP which is associated with a significant increase in the accumulation of pTau in different brain regions. The decrease of TNAP levels/activity is about 30%-51% at 6 hours and 17%-27% at 24 hours. In view of the known function of TNAP in dephosphorylating pTau, the accumulation of pTau after brain injury could be due to the decreased TNAP activity resulting from its decreased levels in the brain after the injury. The decreased activity of TNAP in the brain after injury was associated with a significantly decreased total alkaline phosphatase activity in the plasma which can be used as a biomarker for the diagnosis and prognosis of brain injury. These results suggest that increasing the levels or activity of TNAP in the brain could be a therapeutic strategy against tauopathy/CTE. The levels of TNAP in the brain could be increased by intranasal nose-to-brain delivery of TNAP using a nasal spray and the activity of TNAP in the brain can be increased by intranasal administration of TNAP activators.

What is claimed is:

1) A method of diagnosing traumatic brain injury-induced tauopathy/chronic traumatic encephalopathy (CTE) comprising:
a) obtaining a control sample(s) from a control patients who has not been diagnosed with tauopathy/CTE and recording a normal range of tissue non-specific alkaline phosphatase (TNAP) or total alkaline phosphatase (AP) activity;
b) obtaining a samples from an object patient(s) who have been diagnosed with tauopathy/CTE;
c) comparing the of tissue non-specific alkaline phosphatase (TNAP) or alkaline phosphatase (AP) levels/activities of said object patient(s) to said controls;
d) determining if said object patient(s) have been exposed to traumatic brain injury if said of tissue non-specific alkaline phosphatase (TNAP) or alkaline phosphatase (AP) levels are decreased below the normal range.

2) The method of claim 1, wherein said step b) sample is taken at 3, 6, 12, 24 hours post suspected exposure.

3) The method of claim 1, wherein said sample is a brain tissue sample, blood sample, cerebrospinal fluid, plasma or serum sample.

4) The method of claim 1, wherein said samples were taken from said object patients using devices that detects the extent of decrease in of tissue non-specific alkaline phosphatase (TNAP) or alkaline phosphatase (AP) levels in the above bio-samples after traumatic brain injury.

5) A method of treating traumatic brain injury-induced tauopathy/CTE comprising: administering to a patient a therapeutically effective amount of tissue non-specific alkaline phosphatase (TNAP) or activators of tissue non-specific alkaline phosphatase (TNAP) to the brain by the intranasal route of administration.

6) The method of claim 1, wherein said levels/activities of said of tissue non-specific alkaline phosphatase (TNAP) or alkaline phosphatase (AP) are decreased to a first level at 6 hours compared to the normal range and decreased to a second level at 24 hours compared to said normal range, wherein said first level is more decreased than said second level compared to said normal range.

7) The method of claim 1, wherein said decrease of tissue non-specific alkaline phosphatase (TNAP) or alkaline phosphatase (AP) levels/activity is about 30%-51% at 6 hours and 17%-27% at 24 hours.

8) The method of clam 7, wherein there is a decrease in levels of alkaline phosphatase that occurs in plasma of said object patient that corresponds to the decrease of tissue non-specific alkaline phosphatase (TNAP) in the object patient's brain.

9) A method of diagnosing traumatic brain injury-induced tauopathy/chronic traumatic encephalopathy (CTE) comprising:
a) obtaining a control sample from a control patient who has not been diagnosed with tauopathy/CTE and recording a normal range of tissue alkaline phosphatase (AP);
b) obtaining a sample from an object patient who has been diagnosed with tauopathy/CTE;
c) comparing the AP levels/activities of said object patient to said control;
d) determining if said object patient has been exposed to traumatic brain injury if said AP levels are decreased below the normal range.

10) The method of claim 9, wherein said step b) sample is taken at 3, 6, 12, 24 hours post suspected exposure.

11) The method of claim 9, wherein said sample is a brain tissue sample, blood sample, cerebrospinal fluid, plasma or serum sample.

12) The method of claim 9, wherein said sample is taken from said object patient using devices that detects the extent of decrease in AP levels in the above bio-samples after traumatic brain injury.

13) The method of claim 9, wherein said levels/activities of said AP are decreased to a first level at 6 hours compared to the normal range and decreased to a second level at 24 hours compared to said normal range, wherein said first level is more decreased than said second level compared to said normal range.

14) The method of claim 9, wherein said decrease of AP levels/activity is about 30%-51% at 6 hours and 17%-27% at 24 hours.

15) A method of diagnosing traumatic brain injury-induced tauopathy/chronic traumatic encephalopathy (CTE) comprising:
a) obtaining a control sample from a control patient who has not been diagnosed with tauopathy/CTE and recording a normal range of tissue non-specific alkaline phosphatase (TNAP);
b) obtaining a sample from an object patient who has been diagnosed with tauopathy/CTE;
c) comparing the TNAP levels/activities of said object patient to said control;
d) determining if said object patient has been exposed to traumatic brain injury if said TNAP levels are decreased below the normal range.

15) The method of claim 15, wherein said step b) sample is taken at 3, 6, 12, 24 hours post suspected exposure.

16) The method of claim 15, wherein said sample is a brain tissue sample, blood sample, cerebrospinal fluid, plasma or serum sample.

17) The method of claim 15, wherein said sample is taken from said object patient using devices that detects the extent of decrease in TNAP levels in the above bio-samples after traumatic brain injury.

18) The method of claim 15, wherein said levels/activities of said TNAP are decreased to a first level at 6 hours compared to the normal range and decreased to a second level at 24 hours compared to said normal range, wherein said first level is more decreased than said second level compared to said normal range.

19) The method of claim 15, wherein said decrease of TNAP levels/activity is about 30%-51% at 6 hours and 17%-27% at 24 hours.

20) The method of claim 19, wherein there is a decrease in levels of alkaline phosphatase that occurs in plasma of said object patient that corresponds to the decrease of TNAP in the object patient's brain.
